SEQUENCE LISTING (2 sequences)

SEQ ID NO: 1 - protein, 30 aa, Homo sapiens
KKKQKNGKKHQKKKVLKVRKSQRSRQKKTT

SEQ ID NO: 2 - protein, 30 aa, synthetic construct
  REGION 1..30: note = cxcl9(74-103) variant
  MOD_RES 1: note = ACETYLATION
  MOD_RES 1..3: note = Orn
  MOD_RES 5: note = Orn
  MOD_RES 8..9: note = Orn
  MOD_RES 12..14: note = Orn
  MOD_RES 17: note = Orn
  MOD_RES 20: note = Orn
  MOD_RES 24: note = Orn
  MOD_RES 27..28: note = Orn
XXXQXNGXXHQXXXQQXVRXSQRXRQXXTT

(19) United States
(12) Patent Application Publication
(10) Pub. No.: US 2025/0144177 A1
(43) Pub. Date: May 8, 2025

(54) CXCL9 PEPTIDE FOR THE TREATMENT OF LUNG TISSUE INFLAMMATION IN LUNG INFECTION

(71) Applicants: Katholieke Universiteit Leuven, Leuven (BE); Universidade Federal De Minas Gerais, Minas Gerals (BR)

(72) Inventors: Paul Proost, Leuven (BE); Sofie Struyf, Leuven (BE); Mauro M. Teixeira, Belo Horizonte (BR); Flavio A. Amaral, Minas Gerals (BR); Daiane Boff, New York, NY (US)

(21) Appl. No.: 18/833,585

(22) PCT Filed: Feb. 3, 2023
(86) PCT No.: PCT/EP2023/052629

§ 371 Date: Jul. 26, 2024

(30) Foreign Application Priority Data
Feb. 3, 2022 (EP) 22155028.8

(51) Int. Cl. A61K 38/19; A61P 11/00

(52) U.S. Cl. CPC A61K 38/195; A61P 11/00

(57) ABSTRACT

The invention relates to a peptide of at most 40 amino acids comprising amino acids 74-103 of CXCL9 for use in the treatment or prevention of inflammation of lung tissue in a patient with a bacterial or viral lung infection.

### FIELD OF THE INVENTION

The invention relates to novel medical uses of a peptide comprising the COOH— terminal fragment of the chemokine CXCL9 (CXCL9 (74-103)).

The invention further relates to the treatment or prevention of inflammation of lung tissue in a patient with a lung infection.

### BACKGROUND OF THE INVENTION

Pneumonia is among the leading cause of hospitalization and mortality worldwide According to the World Health Organization (WHO), it accounts for 15% of all deaths of children under 5 years old. Pneumonia is characterized as an acute infection of the lung parenchyma and can be caused by a wide variety of microorganisms, including bacteria, viruses, and fungi. Among bacteria, Klebsiella pneumoniae an encapsulate, non-motile, lactose-fermenting, anaerobic, and gram-negative rod is an important causative agent of hospital-acquired pneumonia (HAP). Pneumonia associated with K. pneumonia is characterized by a severe and exacerbated acute inflammation of lung tissue with important recruitment of neutrophils and the high production of cytokines and chemokines [Friedlander et al. Pathophysiology. 2020, 1-5]. This intense inflammatory response can severely compromise lung function leading to a decrease in compliance and shortness of breath that can progress to respiratory failure and even death.

The recruitment of neutrophils to the lungs during infection is essential for bacterial clearance and to avoid its dissemination. Indeed, depletion of neutrophils increases the bacterial burden in the lungs of mice infected with K. pneumonia [Xiong et al. Infect. Immun. 2015, 83, 3418-3427; Liu et al. J. Immunol. Res. 2020, 2020]. At the site of infection, activated neutrophils play a fundamental role in bacterial killing by a range of antimicrobial tools. They can directly kill K. pneumonia by releasing granules, neutrophil extracellular traps (NETs), antimicrobial peptides, and serine proteases. In addition, neutrophils efficiently phagocyte and kill K. pneumonia by the production of reactive oxygen species (ROS). Besides antimicrobial functions, neutrophils also play a role in the immune response against K. pneumonia by the production of inflammatory cytokines such as TNF, IL-1, IL-17, and IL-12. However, neutrophils can act as a double edged sword because their excessive accumulation and the release of those antimicrobial mediators can cause tissue damage and contributes to the development of severe disease [Craig et al. Infect. Immun. 2009, 77, 568-575].

The roles of neutrophils in the progression of pneumonia make them potential targets of neutralization during microbial infection in general and bacterial infection more specifically. The ideal strategy is attenuating their accumulation and destructive potential while maintaining their critical antibacterial properties. Chemokines play an essential role in the migration of neutrophils from blood to the infected lung and they emerged as a potential target to inhibit neutrophil accumulation [Dyer et al. Glycobiology 2015, 26, 312-326]. At this point, CXC chemokines with ELR (glutamic acid-leucine-arginine) motif (CXCL1, CXCL2, CXCL3, CXCL5, CXCL6, CXCL7 and CXCL8) play a key role in neutrophil recruitment by binding to CXCR1 and/or CXCR2 receptors [Russo et al. Expert Rev. Clin. Immunol. 2014, 10, 593-619]. Chemokines bind to glycosaminoglycans (GAGs) in endothelial cells and generate an immobilized chemokine gradient that directs cell migration. In this context, compounds that compete with chemokines for GAG binding could decrease neutrophil migration to the site of infection. The systemic treatment with a fragment of the chemokine CXCL9 consisting of its 30 COOH-terminal amino acids [CXCL9 (74-103)] successfully inhibits neutrophil migration in murine models of joint inflammation and liver damage.

### SUMMARY OF THE INVENTION

The present invention demonstrates that the therapeutic treatment with CXCL9 (74-103) reduced the massive recruitment of neutrophils to lung tissue and reduced levels in the lungs of the inflammatory cytokine IL-1β without affecting local bacterial burden. The treatment resulted in improved lung function in mice infected with K. pneumonia.

Klebsiella pneumoniae (Kp) is an important pathogen associated with hospital-acquired pneumonia (HAP). Bacterial pneumonia is characterized by a harmful inflammatory response with a massive influx of neutrophils, production of cytokines and chemokines and consequent tissue damage. Targeted therapies to block neutrophil migration to avoid tissue damage while keeping the antimicrobial properties of tissue remains a challenge in the field. The present invention shows the effect of the anti-inflammatory properties of the chemokine fragment CXCL9 (74-103) in pneumonia induced by Kp in mice. Mice were infected by intratracheal injection of Kp and 6 hours after infection treated systemically with CXCL9 (74-103). The recruitment of leukocytes, levels of cytokines and chemokines, colony forming units (CFU) and lung function were evaluated. The treatment with CXCL9 (74-103) decreased neutrophil migration to the airways and the production of the cytokine IL-1β without affecting bacterial control. In addition, the therapeutic treatment improved lung function in infected mice. This indicates that the treatment with CXCL9 (74-103) reduced inflammation and improved lung function in Kp-induced pneumonia.

Bacterial lung infection leads to recruitment of neutrophils which have antibacterial effects but cause inflammation and tissue damage, and thus decrease lung function.

Based on the state of the art it would be assumed that the use of CXCL9 (74-103) in order to prevent tissue damage by decreased neutrophils, also would inhibit the antibacterial activity of neutrophils, leading to a more severe bacterial infection. Surprisingly, this expected and feared increase in bacterial growth is not observed.

One aspect of the invention relates to a peptide comprising amino acids 74-103 of CXCL9 [KKKQKNGKKHQKKKVLKVRKSQRSRQKKTT [SEQ ID NO:1]] for use in the treatment or prevention of inflammation of lung tissue in a patient.

Typically in these patients lung infection is caused by a bacterium, parasite or virus or co-infection with multiple micro-organisms.

The peptide comprising amino acids 74-103 of CXCL9 for use in the claimed invention can have a length of e.g. up to 40 amino acids, or up to 35 amino acids, can have 1 or 2 additional amino acids at the N-terminus, and/or can have 1 or 2 additional amino acids at the C-terminus.

Modified versions for use in the claimed invention include for example replacements of one or more L-amino acids by D-amino acids, replacements of one or more Lysines by Ornithine. Other modifications include one or more additional N-terminal amino acids or addition of chemical groups modifying the N-terminal amino acid to avoid degradation from the N-terminus by aminopeptidases, e.g. an acetyl- or formyl-group, pyroglutamic acid (pQ), a pyroglutamic acid-proline dipeptide (pQ-Pro-).

Other modifications are modifications at the C terminus to avoid degradation by carboxypeptidase e.g. modification or amidation of the —COOH group at the C-terminus into —CO—NH₂.

Other modified versions are peptide dimers on a C-terminal lysine (i.e. Lys with on the alpha and epsilon NH₂-group of lysine the peptide and inclusion of N-terminal (pQ-Pro-) or a dimer on lysine with D amino acids instead of L amino acids.

Specific embodiments are peptides lacking 1, 2, 3, 4, 5, 6, 7, 8, 9, or 10 of the carboxyterminal amino acids of SEQ ID NO: 1.

Further modified versions of CXCL9 (74-103) have Lysine replaced by ornithine, Valine and Leucine in the second GAG-binding motif are replaced by Glutamine and a third GAG-binding motif is created by replacing Serine with a positive amino acid. An example hereof is Acetyl-OOOQONGOOHQOOOQQOVROSQRORQOOTT-amide [SEQ ID NO: 2]

Modified version generally relates to peptides such as those which retain their property to compete with chemokines for GAG binding as disclosed in Crijns et al. Front. Immunol. 2020, 11].

Examples of viral infections are influenza virus, coronavirus and RSV (respiratory syncytial virus).

Examples of parasite infections are roundworms such as Ascaris sp.

Typically the peptide is for use in the treatment or prevention of inflammation of lung tissue in a patient having a bacterial lung infection, such as a lung infection caused by Streptococcus, Klebsiella, Pseudomonas, Staphylococcus, or viral infection such as influenza virus, corona virus, RSV or a combination of bacterial and viral infection. More specifically the bacterial lung infection is caused by K. pneumonia. The bacteria causing lung infection can be antibiotic resistant strains.

The peptide can be administered for example intravenously, or as a spray to the lungs.

The peptide can be administered in combination with other pharmaceutical compounds such as antibiotics.

The invention is further summarised in the following statements:

1. A peptide of up to 40 amino acids, comprising amino acids 74-103 of CXCL9 [KKKQKNGKKHQKKKVLKVRKSQRSRQKKTT [SEQ ID NO: 1]], or a variant thereof for use in the treatment or prevention of inflammation of lung tissue in a patient with a bacterial or viral lung infection,
wherein said variant is selected from the group consisting of:
   one or more D-amino acids,
   one or more substitutions of Lysine in SEQ ID NO: 1 with Ornithine,
   a substitution of Serine in SEQ ID NO: 1 with a charged amino acid, (charged amino acids are Asp, Glu, Arg, Lys and His)
   a substitution of Valine and/or Leucine in SEQ ID NO: 1 with Glutamine,
   a modification of the NH₂ group of the amino terminus of the peptide and/or modification of the COOH group of the carboxyterminus of the peptide,
   a dimer of the peptide, and
   absence of one or two of the Threonine at the C-terminus of SEQ ID NO: 1.

2. The peptide or variant thereof for use according to statement 1, which has a length of up to 35 amino acids.

3. The peptide for use according to statement 1 or 2, comprising the sequence

[SEQ ID NO: 1]
KKKQKNGKKHQKKKVLKVRKSQRSRQKKTT.

4. The peptide for use according to any one of statements 1 to 3, consisting of the sequence KKKQKNGKKHQKKKVLKVRKSQRSRQKKTT [SEQ ID NO: 1].

5. The peptide or variant thereof for use according to any one of statements 1 to 4, in the treatment or prevention of inflammation of lung tissue in a patient with a bacterial lung infection.

6. The peptide or variant thereof for use according to any one of statements 1 to 5, in the treatment or prevention of inflammation of lung tissue in a patient with a gram negative bacterial lung infection.

7. The peptide or variant thereof for use according to any one of statements 1 to 6, wherein the bacterial lung infection is an infection with the gram negative bacterium Klebsiella or Pseudomonas.

8. The peptide or variant thereof for use according to any one of statements 1 to 7, wherein the bacterial lung infection is in an infection by Klebsiella sp.

9. The peptide or variant thereof for use according to statement 8, wherein Klebsiella sp. is K. pneumonia.

10. The peptide or variant thereof for use according to any one of statements 1 to 5, in the treatment or prevention of inflammation of lung tissue in a patient with a gram positive bacterial lung infection.

11. The peptide or variant thereof for use according to any one of statements 1 to 4, wherein the bacterial lung infection is an infection by the gram positive bacterium Staphylococcus or Streptococcus.

12. The peptide or variant thereof for use according to any one of statements 1 to 11, wherein the bacterium is an antibiotic resistant bacterium.

13. The peptide or variant thereof for use according to any one of statements 1 to 4, in the treatment or prevention of inflammation of lung tissue in a patient with a viral infection.

14. The peptide or variant thereof for use according to any one of statements 1 to 5, wherein the peptide is administered intravenously, intraperitoneally or as a spray to the lungs.

15. A method of treating or preventing inflammation of lung tissue in a patient with a bacterial or viral lung infection comprising the step of administering to said patient a peptide of up to 40 amino acids, comprising amino acids 74-103 of CXCL9 [KKKQKNGKKHQKKKVLKVRKSQRSRQKKTT [SEQ ID NO: 1]], or a variant thereof for use in the treatment or prevention of inflammation of lung tissue in a patient with a bacterial or viral lung infection,
wherein said variant is selected from the group consisting of:
   one or more D-amino acids,
   one or more substitutions of Lysine in SEQ ID NO: 1 with Ornithine,
   a substitution of Serine in SEQ ID NO: 1 with a charged amino acid,
   a substitution of Valine and/or Leucine in SEQ ID NO: 1 with Glutamine,
   modification of the NH₂ group of the amino terminus of the peptide and/or modification of the COOH group of the carboxyterminus of the peptide,
   a dimer of the peptide,
   absence of one or two of the Threonine at the C-terminus of SEQ ID NO: 1.

### BRIEF DESCRIPTION OF THE DRAWINGS

FIG. 1: Binding of CXCL9 (74-103) to lung endothelium. Mice were injected intravenously with TAMRA-labelled CXCL9 (74-103) and lung tissues were collected after 6 and 12 hours for confocal analysis. About 5 minutes before euthanasia mice were injected intravenously with APC-labelled anti-CD31 antibodies. (A) Lungs were removed and imaged to evaluate the binding of CXCL9 (74-103) in the lung endothelium and (B) the percentage of TAMRA area fraction was calculated. (C) Higher magnification of colocalization of CD31 and CXCL9 (74-103) 6 hours after the injection. Data are shown as median from one representative out of two independent experiments. #p<0.05 when compared with the 6 hours; n=3-4 mice per group.

FIG. 2: The treatment with CXCL9 (74-103) decreases lung inflammation after LPS instillation. Mice were instilled with LPS (25 μg/mouse) or saline (Ctrl) and after 6 hours mice were treated intravenously with CXCL9 (74-103) 100 μg/100 μL or vehicle (PBS). After 18 hours mice were euthanized and the number of (A) total leukocytes, (B) neutrophils and (C) mononuclear cells was evaluated in BALF. (D) Levels of IL-1β and the chemokines (E) CXCL1, (F) CXCL2, (G) CXCL6 and (H) total protein concentrations were also evaluated in the BALF. Data are shown as median from one representative out of three independent experiments. *p<0.05 when compared to control group; #p<0.05 when compared with the vehicle group; n=5 mice per group. BALF-Bronchoalveolar fluid.

FIG. 3: The treatment with CXCL9 (74-103) improved lung function after LPS instillation. Mice were instilled with LPS (25 μg/mouse) and after 6 hours mice were treated intravenously with CXCL9 (74-103) 100 μg/100 μL or vehicle (PBS). Lung function was evaluated after 24 hours of LPS challenge. (A) Expiratory volume at 20 milliseconds (FEV20), (B) Tiffeneau-Pinelli index (FEV20/FVC), (C) resistance, (D) dynamic compliance, (E) Tidal volume and (F) Minute volume. Data are shown as median from one representative out of three independent experiments. *p<0.05 when compared to control group; #p<0.05 when compared with the vehicle group; n=5 mice per group

FIG. 4: The treatment with CXCL9 (74-103) decreased inflammation after pneumonia induced by Klebsiella pneumoniae. Mice were infected with K. pneumoniae (1×10⁶ CFU/mouse) by intratracheal injection and after 6 hours mice were treated intravenously with CXCL9 (74-103) 100 μg/100 μL or vehicle (PBS). Control mice received a saline injection into the trachea. Mice were euthanized 24 hours after intratracheal challenge and (A) number of total leukocytes, (B) neutrophils and (C) mononuclear cells was evaluated in BALF. (D) Levels of IL-1ß, of the chemokines (E) CXCL1, (F) CXCL2 and (G) CXCL6 and (I) total protein were measured in the BALF. (H) CFU counts in BALF and (J) CFU counts in the lung tissue. Data are shown as median from one representative out of three independent experiments. *p<0.05 when compared to control (Ctrl) group; #p<0.05 when compared with the vehicle (v) group; n=5 mice per group. BALF-Bronchoalveolar fluid.

FIG. 5: The treatment with CXCL9 (74-103) improved lung function after pneumonia induced by Klebsiella pneumoniae. Mice were infected with Klebsiella pneumoniae (1×10⁶ CFU/mouse) by intratracheal injection and after 6 hours mice were treated intravenously with CXCL9 (74-103) 100 μg/100 UL or vehicle (PBS). Lung function was evaluated at 24 hours after challenge. (A) Expiratory volume at 20 milliseconds (FEV20), (B) Tiffeneau-Pinelli index (FEV20/FVC), (C) resistance, (D) dynamic compliance (E) Tidal volume and (F) Minute volume. Data are shown as median from one representative out of three independent experiments. *p<0.05 when compared to the control (Ctrl) group; #p<0.05 when compared with the vehicle (v) group; n=5-8 mice per group.

FIG. 6: CXCL9 (74-103) treatment reduces inflammatory parameters in S. aureus infection and improves clinical score and lung function

(A) Numbers of total leukocytes and (B) neutrophils in BALF were counted as a measurement for the lung inflammation level. (C) Clinical score, (D) Lung Resistance (RI), and (E) Dynamic Compliance Forced (Cdyn)

FIG. 7 CXCL9 (74-103) treatment reduces several inflammatory parameters in mice after infection of the lungs with the MHV-3 corona virus and improves lung function and histopathological score

(A) Numbers of total leukocytes and (B) neutrophils in BALF were counted as indicators of inflammation. (C) Lung Resistance (RI), and (D) Dynamic Compliance Forced (Cdyn). (E) Histopathological score and (F) Contingency graph according to ranges of tissue damage (severe, intense, moderate, mild, and absent) FIG. 8. CXL9 (74-103) has no antibacterial effect on S aureus cultures

### DETAILED DESCRIPTION

The treatment of bacterial pneumonia has been a challenge in the last decade due to, among other factors, antimicrobial resistant strains such as carbapenem-resistant K. pneumonia and the lack of options to prevent the harmful inflammatory response developed in these infections. The present invention investigates the control of excessive lung inflammation and loss of function after K. pneumonia infection in mice by targeting neutrophil accumulation in lung compartments. Herein is demonstrated that the therapeutic treatment with a cationic peptide derived from the chemokine CXCL9 (74-103) reduced the recruitment of neutrophils in murine models of LPS-induced lung inflammation and K. pneumonia-induced pneumonia. In both models, this treatment also reduced the amount of IL-1β in bronchoalveolar fluid without impairing the clearance of K. pneumonia. Importantly, the treatment also improved lung function after these insults. These findings highlight that decreasing neutrophil accumulation in the tissue by controlling the actions of chemokines in the primordial steps for neutrophil diapedesis can prevent excessive tissue inflammation and malfunction.

Neutrophils are essential cells for bacterial elimination during infection. However, their accumulation and secretion of antimicrobial mediators are associated with tissue damage [Boff et al. Eur. J. Immunol. 2018, 48, 454-463; Boff et al. Int. J. Mol. Sci. 2018, 19, 468]. In this context, therapies that target neutrophil migration while maintaining their antimicrobial function are still a main challenge in this field. Some strategies to inhibit neutrophil migration in pulmonary sterile diseases have shown to be effective in different experimental models in mice. For example, the antagonism of the chemokine receptor CXCR2 was able to reduce neutrophil recruitment and improved tissue damage in asthma, in LPS-induced acute inflammation and in bleomycin-induced lung fibrosis. Because of the promising results in animal models, these CXCR2 antagonists are now being tested in clinical trials for lung diseases. Regarding bacterial pneumonia, the inhibition of neutrophil recruitment has controversial outcomes. Some studies evaluated the prophylactic inhibition of neutrophils, as the preventive treatment with G31P, an antagonist of CXCR2, decreasing neutrophil recruitment without affecting bacterial load in pneumonia induced by K. pneumonia. Moreover, the pre-treatment with VAP-1/SSAO, an endothelial bound adhesion molecule with amine oxidase activity that is involved in neutrophil egress from the microvasculature during inflammation, decreased neutrophil migration but increased bacterial load during K. pneumonia-induced pneumonia. In another study, the therapeutic administration of a monoclonal antibody against the granulocyte-colony stimulating factor receptor (G-CSFR mAb) during Streptococcus pneumoniae infection significantly reduced blood and airway neutrophil numbers without affecting bacterial load. Herein, it is demonstrated that the treatment with CXCL9 (74-103) decreased neutrophil migration to the lungs without affecting the bacterial clearance by the host after K. pneumonia infection, even when treatment started 6 hours after infection. The initial recruitment of neutrophils to the lung before application of CXCL9 (74-103) may have an important beneficial effect on the control of K. pneumonia. Therefore, reducing massive neutrophil accumulation by this treatment at later time points may have positive effects to preserve tissue function without stimulation of bacterial growth. In addition, the treatment with CXCL9 (74-103) did not impact the number of total macrophages, which are also important cells for the bacterial killing process.

The high production of cytokines and chemokines can be easily detected systemically and locally and these inflammatory molecules are important biomarkers during the development of acute lung diseases including the current coronavirus disease 2019 (COVID-19) pandemic. Pneumonia induced by K. pneumonia or by LPS are associated with the overproduction of IL-1β and neutrophil-related chemokines such as CXCL1, CXCL2 and CXCL6.

Here, mice treated with CXCL9 (74-103) presented with a substantial reduction of IL-1β concentrations in BALF in pneumonia induced by both K. pneumonia or LPS. Overexpression of IL-1β in lung tissue coincided with increased lung vascular permeability, associated to a massive cellular influx and tissue damage. Activated macrophages represented the main source of IL-1β during acute inflammation and blocking their activation or directly antagonizing IL-1β contributed to a reduction of tissue inflammation in experimental models of pneumoniae. Here, the treatment with CXCL9 (74-103) did not reduce macrophage accumulation in BALF, but significantly decreased the number of neutrophils. At this point, some studies demonstrated that neutrophils are necessary for IL-1β production by macrophages, which may explain the lower detection of this cytokine in CXCL9 (74-103)-treated mice. In addition, neutrophils can also be a source of IL-1β during acute inflammation. Thus, in general, these data explain the direct correlation between the reduction of accumulated neutrophils with the low levels of IL-1β after LPS or K. pneumonia stimulation upon CXCL9 (74-103) treatment.

During acute inflammation, several chemoattractant molecules are produced locally to guide neutrophil migration to the affected tissue. GAGs have a crucial role in the initial steps of leukocyte migration due to their negatively charged structures, retaining positively charged chemokines on endothelium. Thus, endothelium-attached chemokines may bind to their chemokine receptors on the rolling cells, modify integrin conformation from an inside to outside manner on leukocytes and strengthen leukocyte adhesion to endothelial cells. At this point, CXCL9 (74-103) impairs leukocyte migration by competing with chemokine binding to different types of GAGs [Vanheule et al. Front. Immunol. 2017, 8, 1-14; Vanheule et al. J. Biol. Chem. 2015, 290, 21292-21304; Marques et al. Hepatol. Commun. 2021, 0, 1-18]. As a consequence, CXCL9 (74-103) treatment caused a reduction of neutrophil migration to the tissue after LPS and K. pneumonia insults, reducing lung inflammation as exemplified by the lower detection of IL-1β. However, this treatment didn't completely abolish the accumulation of neutrophils in BALF. Thus, the presence of neutrophils, even in lower numbers when compared to non-treated mice, still contribute to the clearance of K. pneumonia once activated by locally produced chemokines. At this point, chemokines not only guide the cells to the site of infection but have an important role in the activation of these cells to deal with infection. A previous study shows that CXCL8 stimulation assisted in the clearance of Staphylococcus aureus by purified neutrophils. Here, the treatment with CXCL9 (74-103) did not reduce the levels of some CXCR2-binding chemokines, which may help to explain why this treatment did not impair the bacterial control.

The lung is the main organ of the respiratory system and its well-functioning is essential to facilitate gas exchange between the environment and bloodstream. Any disturbance of lung homeostasis as occurs in acute inflammation potentially alters its function, which represent an important clinical concern. Besides the direct involvement of IL-1β causing lung damage and dysfunction, exudative fluids and excessive accumulation of neutrophils in lung tissue also play critical roles in lung inflammation, damage and dysfunction during pneumonia. As a result, air space does not inflate properly at higher transpulmonary pressures, resulting in the reduction of the total lung volume and compliance that cause the shortness of breath and low oxygen supply. Targeting chemokines and neutrophil migration have been shown as promising strategies to recover lung function after different types of lung insults, in sterile and viral lung inflammation. Here, LPS and K. pneumonia caused profound alterations in respiratory mechanics in mice and the treatment with CXCL9 (74-103) ameliorates some of those parameters, highlighting its beneficial effects to control critical symptoms of acute lung inflammation.

In conclusion, targeting pulmonary GAGs may be an effective strategy to control signs and symptoms of acute lung inflammation, preventing excessive leukocyte accumulation in pulmonary compartments, reducing local production of pro-inflammatory molecules and ameliorating lung function. In addition, given therapeutically, CXCL9 (74-103) did not abolish neutrophil accumulation in lung tissue, which helps to explain why there was no loss in bacterial control in the case of K. pneumonia infection.

### EXAMPLES

### Example 1. CXCL9 (74-103) Binds to the Lung Endothelium In Vivo

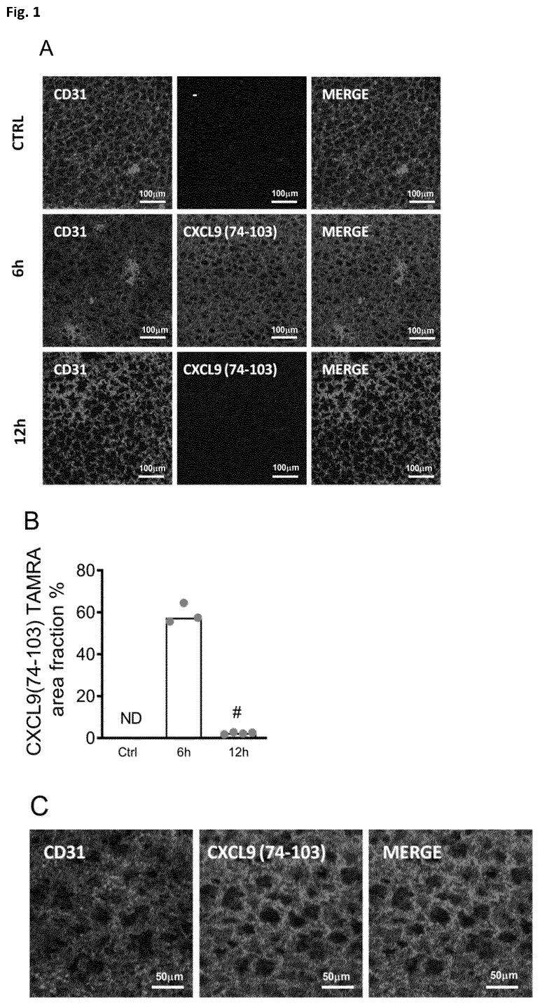

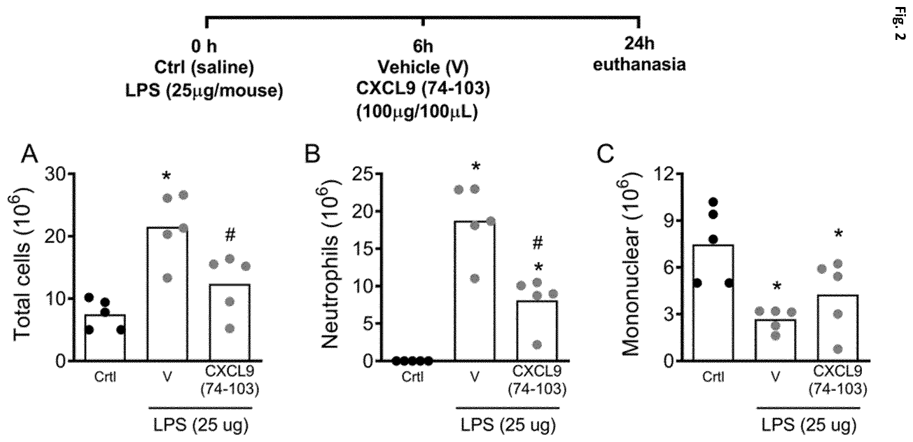

CXCL9 (74-103) is a fragment of the chemokine CXCL9 consisting of the 30 C-terminal amino acids. It was demonstrated that this highly positively charged fragment competes with chemokines for GAG binding [Crijns et al cited above]. To evaluate if CXCL9 (74-103) could bind to GAGs on lung endothelium in vivo, the peptide was site-specifically labelled at the NH₂-terminus with the fluorophore 5 (6) carboxy-tetramethylrhodamine (TAMRA). Mice were injected intravenously with the labelled peptide and an anti-CD31 antibody as a marker for blood vessels. Mice were euthanized 6 or 12 hours after the injection. The lungs were removed and imaged by intravital microscopy. CXCL9 (74-103) was detected in lung tissue only 6 hours after the systemic injection as demonstrated by its colocalization with blood vessels (FIG. 1A-C). Since no sign of CXCL9 (74-103) was detected 12 hours after injection, these data indicate that CXCL9 (74-103) can regulate neutrophil diapedesis to the lung for at least 6 hours.

### Example 2. The Treatment with CXCL9 (74-103) Decreases Lung Inflammation and Neutrophil Recruitment in Response to LPS In Vivo

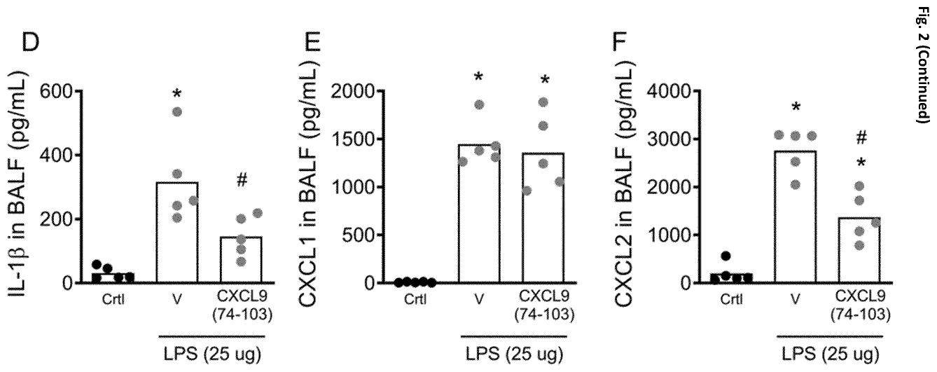

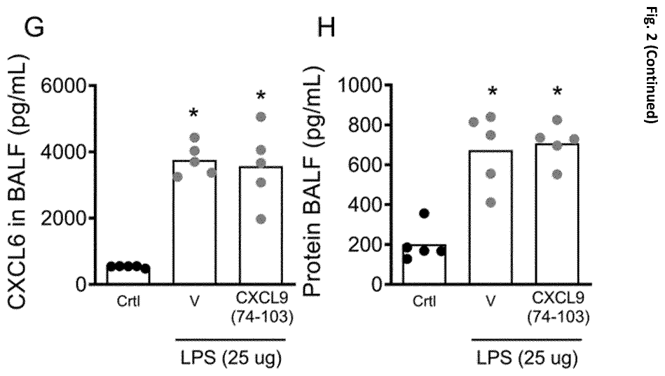

The intranasal administration of lipopolysaccharide (LPS) is known to trigger an inflammatory response characterized by a huge neutrophil accumulation into the airways. It was further evaluated if by binding to GAGs in the lungs, CXCL9 (74-103) could decrease neutrophil migration and inflammation after LPS challenge. Mice were instilled with LPS (25 μg in saline/mouse) and were treated with CXCL9 (74-103) 100 μg/100 μL or vehicle (PBS) intravenously 6 hours later, with the euthanasia occurring 18 hours after the treatment (24 hours after LPS challenge) (FIG. 2). The negative controls were instilled with saline. The treatment with CXCL9 (74-103) was able to decrease the number of total leukocytes and neutrophils recruited to the airways (FIG. 2A-B) but did not affect the recruitment of mononuclear cells (FIG. 2C), mostly composed of macrophages. In addition, the treatment with CXCL9 (74-103) decreased the levels of IL-1β and CXCL2 (FIGS. 2D and F), but no differences were observed on levels of the chemokines CXCL1 and CXCL6 or total protein concentrations in BAL fluid (FIGS. 2E, G and H). Taken together these data demonstrated that the therapeutic treatment with CXCL9 (74-103) controls lung inflammation by reducing neutrophil accumulation and decreasing IL-1β and CXCL2 production in the tissue.

### Example 3. The Treatment with CXCL9 (74-103) Improved Lung Function in LPS-Instilled Mice

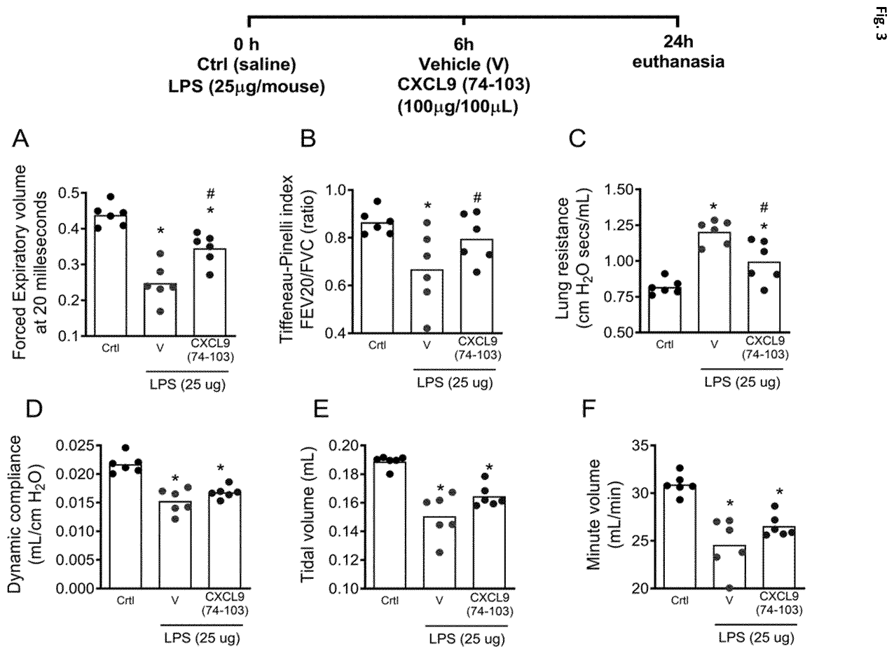

The inflammation associated with intranasal administration of LPS induces alterations in lung capacity and lung mechanics. Since the treatment with CXCL9 (74-103) decreased tissue inflammation, it was next investigated if the treatment would improve lung function. To evaluate airflow limitation, expiratory volume at 20 milliseconds (FEV20) and Tiffeneau-Pinelli index (FEV20/FVC) were analysed. Other parameters evaluated were resistance (measurement of the resistance of the respiratory tract to the airflow movement during normal aspiration and expiration), dynamic compliance (measurement of lung elastic properties) and lung volume measured by Tidal volume (amount of air that can be inhaled or exhaled during one respiratory cycle) and minute volume (amount of gas inhaled or exhaled in one minute). The treatment with CXCL9 (74-103) 6 hours after the LPS instillation improved FEV20, Tiffeneau-Pinelli index and lung resistance when compared to the vehicle-treated group (FIG. 3A-C). However, the treatment was not able to improve significantly the dynamic compliance, Tidal and minute volume (FIG. 3D-F) when compared to vehicle-treated group. Altogether, these findings show a direct association of the reduced lung inflammation with improved lung function in LPS-instilled mice treated with CXCL9 (74-103).

### Example 4. The Treatment with CXCL9 (74-103) Decreased the Inflammatory Response in Pneumonia-Induced by Klebsiella pneumonia

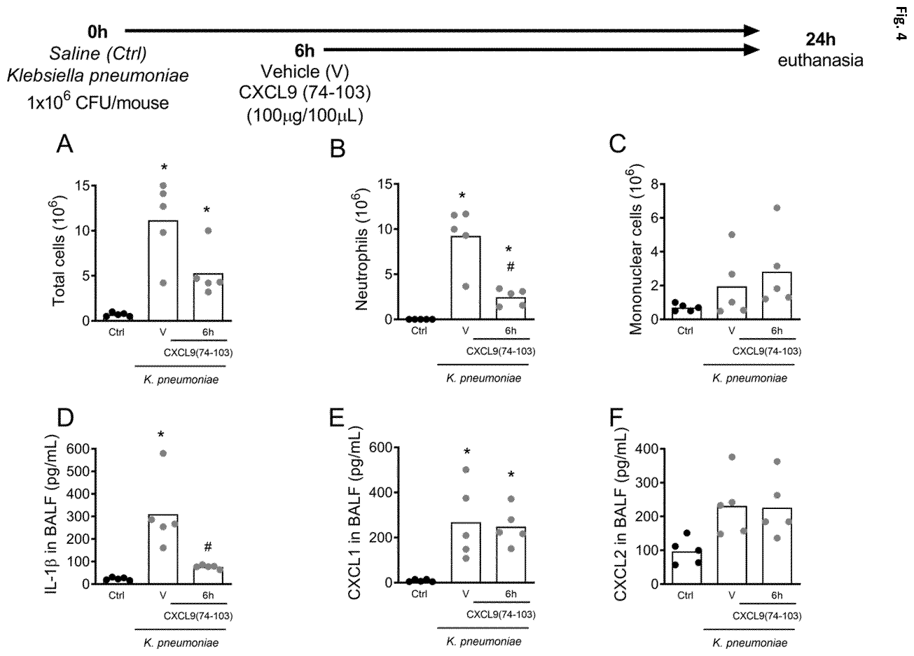

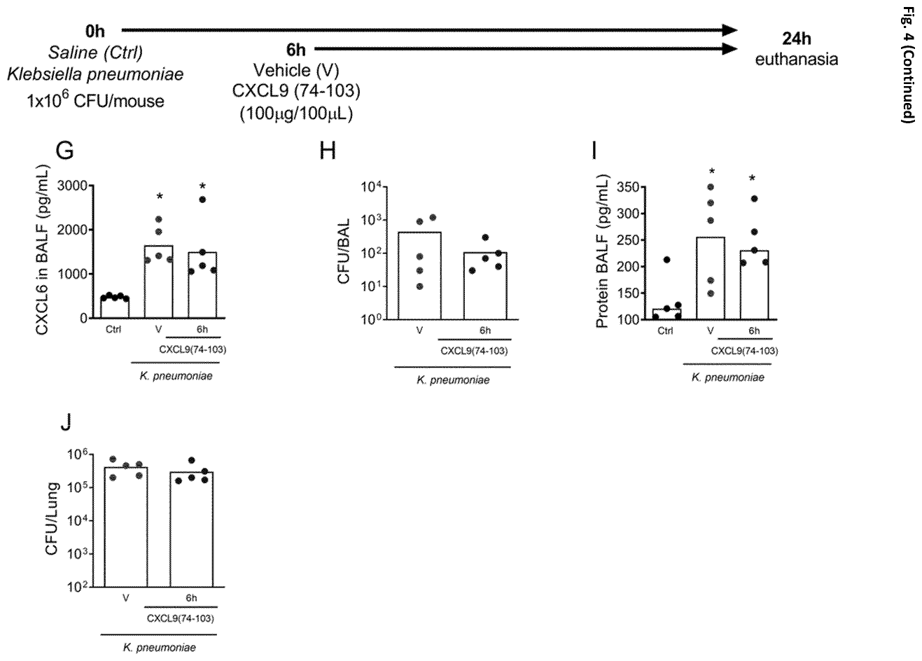

Since the treatment with CXCL9 (74-103) decreased LPS-induced lung inflammation, as represented by reduced neutrophil recruitment and improved lung function, it was next tested if the therapeutic treatment with CXCL9 (74-103) would reduce lung inflammation and function in a model of pneumonia induced by K. pneumonia. Mice were intratracheally infected with K. pneumonia and the treatment with CXCL9 (74-103) or vehicle (PBS) occurred intravenously 6 hours later (FIG. 4). Mice were euthanized 24 hours after the infection for the analysis. As shown in LPS-challenged mice, the treatment with CXCL9 (74-103) decreased the number of total leukocytes and neutrophils recruited to the airways when compared to vehicle-treated mice (FIG. 4A-B). The treatment did not affect the recruitment of mononuclear cells (FIG. 4C). In addition, the treatment with CXCL9 (74-103) reduced the levels of IL-1β compared to vehicle-treated mice (FIG. 4D) while no difference was found in the levels of the chemokines CXCL1, CXCL2, and CXCL6 and total protein in BAL fluid (FIGS. 4E-G and 4I). Considering neutrophils as essential cells for bacterial clearance, it was investigated if this treatment could affect bacterial burden in lungs. Bacteria CFU were determined in the airways (in BAL fluid) and in the lung tissue. The treatment did not increase bacterial load in both compartments as compared to the vehicle-treated group. (FIGS. 4H and 4I). These results indicate that the therapeutic treatment with CXCL9 (74-103) reduced lung inflammation without affecting host bacterial control.

### Example 5. The Treatment with CXCL9 (74-103) Improved Lung Function in Pneumonia Induced by Klebsiella pneumonia

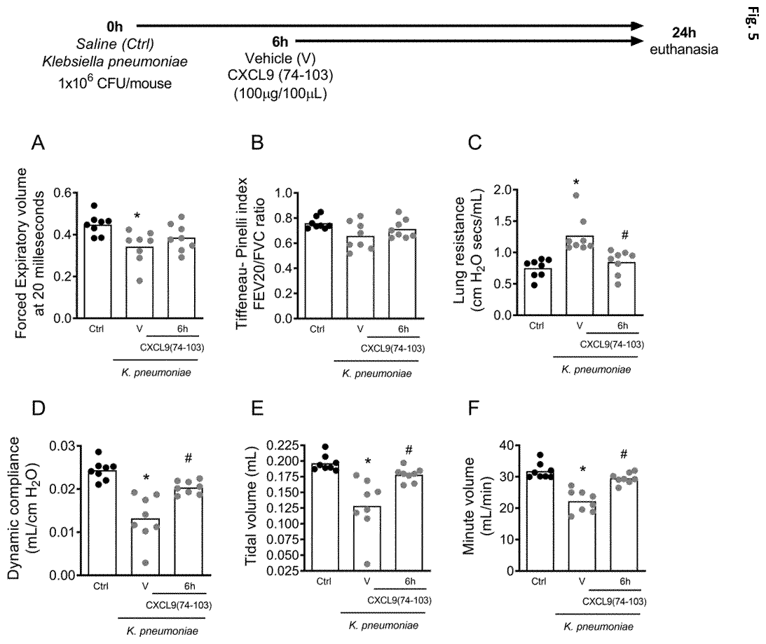

Similar to LPS-induced acute inflammation, pneumonia induced by K. pneumonia also promotes alterations in lung function (FIG. 5). It was tested whether the therapeutic treatment with CXCL9 (74-103) could improve lung function (FIG. 5). Although this treatment did not affect FEV20 and Tiffeneau-Pinelli index (FEV20/FVC) significantly (FIG. 5A-C), treatment with CXCL9 (74-103) 6 hours after K. pneumonia infection improved dynamic compliance, minute volume, and lung resistance when compared with vehicle-treated mice (FIG. 5D-F). These results indicated that the therapeutic treatment with CXCL9 (74-103) improved lung function after pneumonia induced by K. pneumonia.

### Example 6 CXCL9 (74-103) Treatment Reduces Inflammatory Parameters in S. aureus Infection and Improves Clinical Score and Lung Function

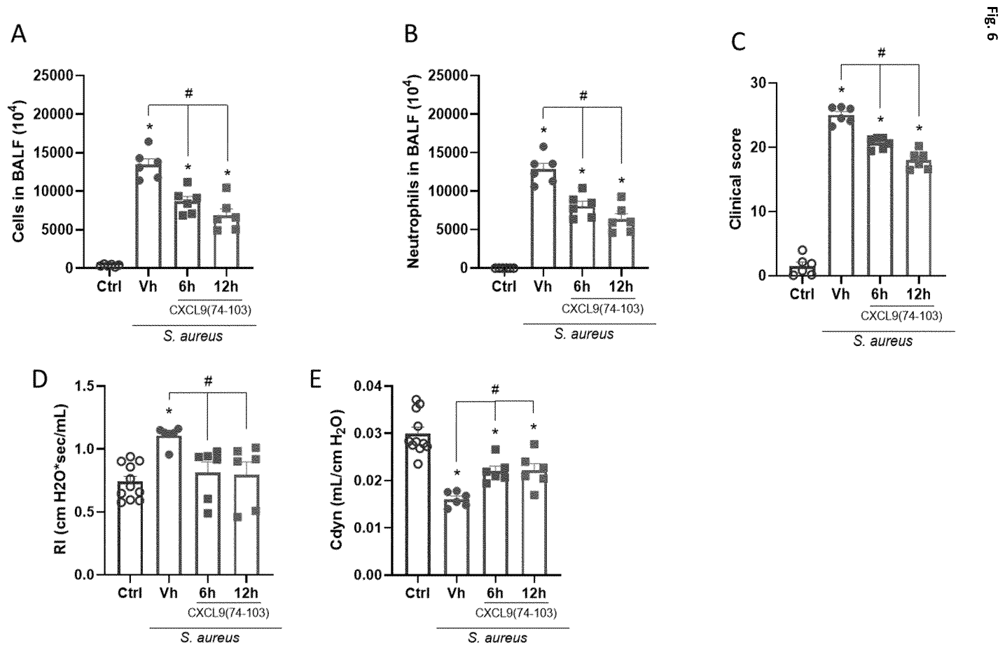

C57BL/6 mice were infected intranasally with S. aureus (10⁸ CFU/mouse) or saline (Ctrl group) and dissected 24 hours later. At the indicated times (6 h or 12 h) after the challenge with bacteria, mice were treated intravenously with 100 μl CXCL9 (74-103) at 1 mg/ml. FIG. 6 shows: (A) Numbers of total leukocytes and (B) neutrophils in BALF were counted as a measurement for the lung inflammation level. (C) Clinical score (D) was calculated based on observational parameters and changes in body weight. Right before euthanasia, invasive forced spirometry was performed to investigate functional modifications in pulmonary elasticity represented by (D) Lung Resistance (RI), and (E) Dynamic Compliance Forced (Cdyn); Data are shown as mean±SEM. Each symbol represents data of an individual mouse. *p<0.05 when compared with the healthy, unchallenged control group. #p<0.05 when comparing different time points with the vehicle. ANOVA test followed by Bonferroni correction was used in the graphs with normal distribution. Otherwise, Kruskal-Wallis with Dunn's multiple comparisons test was used. n=6.

### Example 7 CXCL9 (74-103) Treatment Reduces Several Inflammatory Parameters in Mice after Infection of the Lungs with the MHV-3 Corona Virus and Improves Lung Function and Histopathological Score

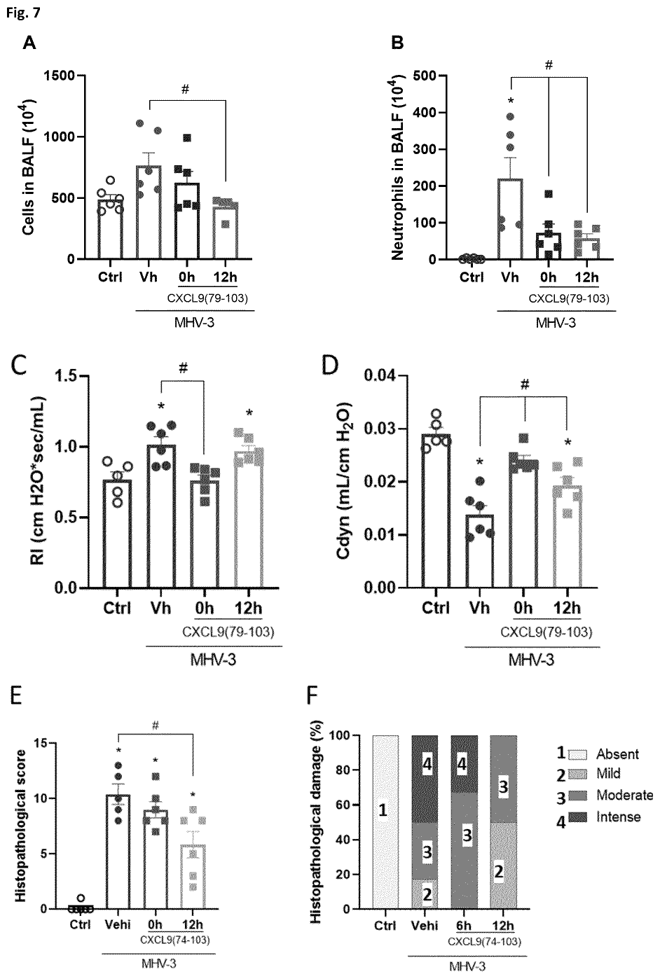

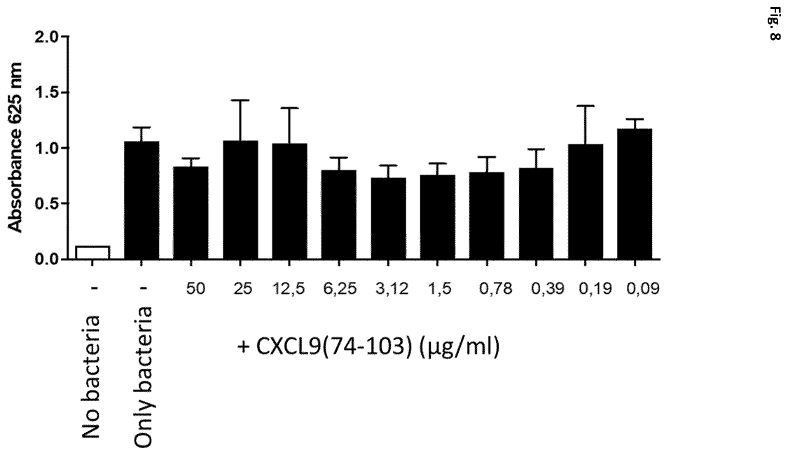

C57BL/6 mice were infected intranasally with the murine coronavirus MHV-3 (3×103 PFU/mouse) or saline (Ctrl group) and dissected 3 days later. At the indicated times after the challenge (0 h or 12 h), mice were treated intravenously with 100 μl CXCL9 (74-103) at 1 mg/ml. FIG. 7 shows (A) Numbers of total leukocytes and (B) neutrophils in BALF were counted as indicators of inflammation. Right before euthanasia, pulmonary mechanic functions were assessed. Invasive forced spirometry was performed to investigate functional modifications in pulmonary elasticity represented by (C) Lung Resistance (RI), and (D) Dynamic Compliance Forced (Cdyn). (E) Histopathological score and (F) Contingency graph according to ranges of tissue damage (severe, intense, moderate, mild, and absent). (A-E) Data are shown as mean±SEM. Each symbol represents data of an individual mouse. *p<0.05 when compared with the healthy, unchallenged control group. #p<0.05 when comparing different time points with the vehicle. ANOVA test followed by Bonferroni correction was used in the graphs with normal distribution. Otherwise, Kruskal-Wallis with Dunn's multiple comparisons test was used. n=6.

### Example 8. CXL9 (74-103) has No Antibacterial Effect

To a culture of S. aureus the peptide CXL9 (74-103) has been added in concentrations ranging from 0.09 μg/ml to 50 μg/ml. No antibacterial effect of the peptide was observed (see FIG. 8).

### Example 9. Resistance of Peptides to Proteolytic Degradation

The following peptides were compared for resistance against proteolytic degradation: CXCL9 (74-103) with L-amino acids (=L-amino acid peptide); CXCL9 (74-103) with D-amino acids (=D-amino acid peptide) and Acetyl-OOOQONGOOHQOOOQQOVROSQRORQOOTT [SEQ ID NO:2] This is a peptide derived from CXCL9 (74-103) with an acetyl-group on the N-terminus, lysines replaced by ornithine and VL (at positions 88 and 89) replaced by QQ and serine (at position 97) also replaced by ornithine (=ornithine peptide).

Incubations were performed with peptides at 50 ng/ml and 37° C. for 5 minutes up to 24 hours with trypsin, plasmin or cathepsin G. After the incubation, the reaction was stopped through addition of trifluoroacetic acid (to reach a final pH<4) and purified by nano-LC with on-line detection of eluted peptides by mass spectrometry.

Sample buffer for incubations with trypsin: 100 mM Tris-HCl pH 8.5; final trypsin (from Sigma) conc. 250 ng/ml

Sample buffer for incubations with plasmin: 50 mM Tris-HCl 0.5 M NaCl PH 7.4; final plasmin conc. 50 μg/ml Sample buffer for incubations with cathepsin G: 20 mM Tris-HCl+1 mM CaCl₂ pH 7.4; final conc. Cathepsin G at 13 mU/litre.

L-amino acid peptide was degraded with trypsin after 5 minutes; partial degraded with plasmin after 5 min, and fully degradation after 30 minutes; the peptide is cathepsin G stable up to 2 h, while more than 90% is degraded after 4 h.

The Ornithine peptide was cleaved at arginine after 5 min with trypsin; cleavage after arginine (<10%) starts with plasmin after 5 min, and >70% was degraded after 4 h incubation with plasmin; no cleavage with cathepsin G after 5 min up to 4 h incubation.

D-amino acid peptide was fully resistant to trypsin at incubations from 5 min. up to 4 hours and resistant to plasmin at incubations between 5 min and 8 hours. The peptide was resistant (less than 5% degradation) to cathepsin G at incubations up to 8 h and 68% degradation after 24 h.

The natural peptide with L-amino acids is highly sensitive to the proteases trypsin and plasmin and upon prolonged incubation also degraded by cathepsin G.

The peptide in which lysine are replaced by ornithine is still rapidly cleaved with trypsin after arginine, cleavage with plasmin is slower compared to the L-amino acid peptide and no cleavage is observed with cathepsin G.

The peptide with D-amino acids is highly resistant to cleavage by trypsin or plasmin and is slowly degraded with cathepsin G.

### Example 10. Materials and Methods

### Mice and Reagents

Eight-to-ten-week-old female C57BL/6J mice were purchased from Centro de Bioterismo da Universidade Federal de Minas Gerais (UFMG). All animals were maintained with filtered water and food ad libitum and kept in a controlled environment. Experiments received prior approval by the animal ethics committee of UFMG (CEUA 347/2019). LPS (Lipopolysaccharide from Escherichia coli serotype O: 111: B4) were purchased from Sigma-Aldrich (St. Louis, MO, USA). The anti-mouse CD31 APC antibody was purchased from Becton Dickinson (BD, Madrid, Spain).

### Solid-Phase Synthesis of the C-Terminal CXCL9-Derived Peptide

The C-terminal peptide of CXCL9, CXCL9 (74-103), was chemically synthesized with fluorenyl methoxycarbonyl (Fmoc) chemistry using an Activo-P11 automated synthesizer (Activotec, Cambridge, UK). Part of the material was fluorescently labeled site-specifically at the N-terminus using TAMRA (Merck Millipore, Darmstadt, Germany). After synthesis, intact synthetic peptides were purified by RP-HPLC and identified by mass spectrometry (Amazon SL or Amazon Speed ETD ion trap mass spectrometers, Bruker, Bremen, Germany).

### Intravital Microscopy

For intravital imaging, mice were injected i.v. with 100 μg/100 μL of TAMRA-labelled CXCL9 (74-103) and 10 minutes prior euthanasia injected with anti-CD31 APC. After 6 and 12 hours of peptide injection, the lungs were removed and imaged using a Nikon Eclipse Ti microscope with a C2 confocal head equipped with three different lasers (excitation at three wavelengths: 405 nm, 488 nm, and 543 nm) and emission bandpass filters at 450/50, 515/30, and 584/50 nm. The z-position was controlled by an automated device and 10× objectives were used on the required resolution. The analysis was performed using Volocity 6.3 software (PerkinElmer).

### Bacterial Strain

The bacterium Klebsiella pneumoniae ATCC 27736 has been kept at the Department of Microbiology (UFMG), and its pathogenicity was stimulated by 10 passages in C57BL/6 mice. Bacteria were frozen in the logarithmic phase of growth and kept at −80° C. at a concentration of 1×10⁹ CFU/mL in tryptic soy broth (Difco, Detroit, MI, USA) containing 10% (vol/vol) glycerol until use.

### LPS-Induced Acute Lung Inflammation

Mice were anaesthetized with ketamine-xylazine (50 mg/mL and 0.02 mg/ml; i.p.) and 30 UL of saline or LPS (25 μg per mouse) was instilled intranasally. Groups of mice were treated with CXCL9 (74-103) 100 μg/100 UL or saline i.v. 6 h after the instillation. At 24 h after the LPS instillation, mice were killed by anaesthetic overdose.

Klebsiella pneumoniae Lung Infection

Bacteria were cultured for 20 h at 37° C. prior to inoculation. The concentration of bacteria in broth was routinely determined by serial 1:10 dilutions. A total of 100 UL of each dilution was plated onto MacConkey agar (Difco) and incubated for 24 h at 37° C. before colonies were counted. Each animal was anesthetized i.p. with 0.2 mL of a solution containing xylazine (0.02 mg mL), ketamine (50 mg mL) and saline in a relative composition of 1:0.5:3. The trachea were exposed and 25 μL of a suspension containing 1×10⁶ CFU of K. pneumonia or saline was administered with a 26-gaugue needle. The skin was closed with surgical staples. Groups of mice were treated with CXCL9 (74-103) 100 μg/100 UL or saline i.v. 6 or 12 h after the infection. At 24 h after the challenge, mice were killed by anaesthetic overdose.

### Bronchoalveolar Lavage Fluid (BALF) and CFU Counts

Mice were euthanized with a lethal solution of ketamine/xylazine (180 and 12 mg/kg, respectively), and BALF was collected by inserting and collecting three times 0.5 mL aliquots of phosphate-buffered saline (PBS), through a 1.7-mm catheter in a 1-mL syringe. After centrifugation, cell pellets were used for total and differential leukocyte counts. The number of total leukocytes was determined by counting them in a modified Neubauer chamber after staining with Turk's solution. Differential counts were obtained from cytospin preparations (Shandon III) by evaluating the percentage of each leukocyte on a slide stained with May-Grünwald-Giemsa stain and examined by light microscopy. BALF supernatants were used for cytokines, chemokines measurements. The bacterial load in the lungs and BALF was accessed by serial dilutions (1:10) in sterile saline and 10 μl of each dilution was plated onto MacConkey agar and incubated for 24 h at 37° C. for determining the number of CFU.

### Measurement of Chemokines, Cytokines

The cytokine IL-1B and the chemokines CXCL1, CXCL2 and CXCL6 were measured from the BALF supernatants by ELISA in accordance with the manufacturer's instructions (R&D Systems).

### Assessment of Respiratory Mechanic Dysfunction

Mice were tracheostomized, placed in a body plethysmograph, and connected to a computer-controlled ventilator (Forced Pulmonary Maneuver System; Buxco Research Systems, Wilmington, NC). Under mechanical respiration the dynamic compliance and lung resistance were determined by resistance and compliance test.

To measure functional residual capacity (FRC), ventilation was stopped at the end of expiration and spontaneous breathing manoeuvres with consequent pressure changes were recorded to calculate the FRC by Boyle's law. To measure the chord compliance and inspiratory capacity (IC), the quasistatic pressure-volume manoeuvre was performed, which inflates the lungs to a standard pressure of +30 cm H₂O and then slowly exhales until a negative pressure of −30 cm H₂O is reached. Chord compliance was determined at the pressure+10 cmH₂O. The total lung capacity was calculated by FRC+IC. Fast-flow volume manoeuvre was performed, and lungs were first inflated to +30 cm H₂O and immediately afterwards connected to a highly negative pressure to enforce expiration until −30 cm H₂O. The forced vital capacity, forced expiratory volume at 50 and 100 ms, and flow-volume curve were recorded during this manoeuvre. Suboptimal manoeuvres were rejected and for each test in every single mouse at least three acceptable manoeuvres were conducted to obtain a reliable mean for all numeric parameters.

### Statistical Analyses

Data were expressed as median and analysis was performed using the statistical software GraphPad Prism 8.0 (GraphPad Software, San Diego, CA). Differences between means were evaluated using ANOVA test, followed by Newman-Keuls. Results with P<0.05 were considered significant.

1. A peptide of up to 40 amino acids, comprising amino acids 74-103 of CXCL9 [KKKQKNGKKHQKKKVLKVRKSQRSRQKKTT [SEQ ID NO:1]], or a variant thereof for use in the treatment or prevention of inflammation of lung tissue in a patient with a bacterial or viral lung infection,
wherein said variant is selected from the group consisting of:
one or more D-amino acids,
one or more substitutions of Lysine in SEQ ID NO: 1 with Ornithine,
a substitution of Serine in SEQ ID NO: 1 with a charged amino acid,
a substitution of Valine and/or Leucine in SEQ ID NO: 1 with Glutamine,
a modification of the NH2 group of the amino terminus of the peptide and/or modification of the COOH group of the carboxyterminus of the peptide,
a dimer of the peptide, and
absence of one or two of the Threonine at the C-terminus of SEQ ID NO: 1.

2. The peptide or variant thereof for use according to claim 1, which has a length of up to 35 amino acids.

3. The peptide for use according to claim 1 or 2, comprising the sequence
[SEQ ID NO: 1]
KKKQKNGKKHQKKKVLKVRKSQRSRQKKTT.

4. The peptide for use according to any one of claims 1 to 3, consisting of the sequence KKKQKNGKKHQKKKVLKVRKSQRSRQKKTT [SEQ ID NO: 1].

5. The peptide or variant thereof for use according to any one of claims 1 to 4, in the treatment or prevention of inflammation of lung tissue in a patient with a bacterial lung infection.

6. The peptide or variant thereof for use according to any one of claims 1 to 5, in the treatment or prevention of inflammation of lung tissue in a patient with a gram negative bacterial lung infection.

7. The peptide or variant thereof for use according to any one of claims 1 to 6, wherein the bacterial lung infection is an infection with the gram negative bacterium Klebsiella or Pseudomonas.

8. The peptide or variant thereof for use according to any one of claims 1 to 7, wherein the bacterial lung infection is in an infection by Klebsiella sp.

9. The peptide or variant thereof for use according to claim 8, wherein Klebsiella sp. is K. pneumonia.

10. The peptide or variant thereof for use according to any one of claims 1 to 5, in the treatment or prevention of inflammation of lung tissue in a patient with a gram positive bacterial lung infection.

11. The peptide or variant thereof for use according to any one of claims 1 to 4, wherein the bacterial lung infection is an infection by the gram positive bacterium Staphylococcus or Streptococcus.

12. The peptide or variant thereof for use according to any one of claims 1 to 11, wherein the bacterium is an antibiotic resistant bacterium.

13. The peptide or variant thereof for use according to any one of claims 1 to 4, in the treatment or prevention of inflammation of lung tissue in a patient with a viral infection.

14. The peptide or variant thereof for use according to any one of claims 1 to 13, wherein the peptide is administered intravenously, intraperitoneally or as a spray to the lungs.

15. A method of treating or preventing inflammation of lung tissue in a patient with a bacterial or viral lung infection comprising the step of administering to said patient a peptide of up to 40 amino acids, comprising amino acids 74-103 of CXCL9 [KKKQKNGKKHQKKKVLKVRKSQRSRQKKTT [SEQ ID NO: 1]], or a variant thereof for use in the treatment or prevention of inflammation of lung tissue in a patient with a bacterial or viral lung infection, wherein said variant is selected from the group consisting of:
one or more D-amino acids,
one or more substitutions of Lysine in SEQ ID NO: 1 with Ornithine,
a substitution of Serine in SEQ ID NO: 1 with a charged amino acid,
a substitution of Valine and/or Leucine in SEQ ID NO: 1 with Glutamine,
modification of the NH₂ group of the amino terminus of the peptide and/or modification of the COOH group of the carboxyterminus of the peptide,
a dimer of the peptide,
absence of one or two of the Threonine at the C-terminus of SEQ ID NO: 1.
